(19) United States
(12) Patent Application Publication
(10) Pub. No.: US 2022/0137012 A1
(43) Pub. Date: May 5, 2022

(54) METHOD FOR ANALYZING DIFFERENTIATION OF METABOLITES IN URINE SAMPLE BETWEEN DIFFERENT GROUPS

(71) Applicant: Korea University Research and Business Foundation, Seoul (KR)

(72) Inventors: Kyoung Heon KIM, Seoul (KR); Hoon Suk CHA, Seoul (KR); Joong Kyong AHN, Seoul (KR); Jung Yeon KIM, Seoul (KR)

(73) Assignee: Korea University Research and Business Foundation, Seoul (KR)

(21) Appl. No.: 17/432,734

(22) PCT Filed: Feb. 21, 2020
(86) PCT No.: PCT/KR2020/002542

§ 371 Date: Oct. 25, 2021

(30) Foreign Application Priority Data
Feb. 22, 2019 (KR) 10-2019-0021461

(51) Int. Cl. G01N 30/72; G01N 30/86; G01N 30/14

(52) U.S. Cl. CPC G01N 30/7206; G01N 30/861; G01N 2030/8813; G01N 30/8637; G01N 30/14

(57) ABSTRACT

The present invention relates to a method for metabolite sampling and analysis for reproducibly sampling as many metabolites as possible in a urine sample without changing to metabolites. The method has effects of presenting a biomarker detection method according to the sex or the like, by establishing optimal conditions for metabolite sampling in urine samples and presenting a metabolite comparison analysis method between different groups on the basis of the optimal conditions.

### TECHNICAL FIELD

The present invention relates to a method for analysis of differences between different groups in a urine sample.

### BACKGROUND ART

Urine is a biological sample most useful for health examination. A urine sample can be conveniently and non-invasively collected and typically contains a lot of various metabolites, so that it can be routinely used for disease diagnosis. Diseases such as diabetes, gout, proteinuria, and specific physiological changes such as pregnancy may change the secretion of metabolites in the body and a constitutional composition of metabolites contained in urine. Therefore, studies to find metabolites in urine specifically altered due to disease and physiological variation and to quantify the same so as to propose biomarkers have been extensively executed for a long time. As such, the study of changes in metabolites due to varied specific states is called metabolomics.

With regard to metabolomic research, it is very important to prevent the change of metabolites in a sample and reproducibly extract as many substances as possible without alteration. In the case of urine metabolomics, a standardized urine metabolite extraction method has been proposed in Nature Protocol (Chan E C et al., 2011, Nat. Protoc. Vol. 6, pp 1483-1499). However, this extraction method is not based on experimental studies and cannot be an optimal urine metabolite extraction method because it refers to and summarizes only the existing methods that have been used previously. The standardized urine metabolite extraction method adopts urease treatment to remove urea in urine, and then conducts protein precipitation and metabolite extraction by administering methanol. However, since urease treatment includes reaction at 37° C. for 1 hour, the metabolites in urine may be modified by activity of enzymes or the like in urine, which in turn possibly deteriorates the ability to discover biomarkers in urine metabolomic studies to discover biomarkers for diagnosis of diseases. In addition, pure methanol has not been compared to and analyzed with other extraction solvents in terms of extraction efficiency and reproducibility, and may not be determined as an optimal extraction solvent. Therefore, it is required to study effects of the urease treatment on the existing standardization method while comparing and analyzing different extraction solvents, and therefore, to suggest a new and optimal extraction method capable of reproducibly extracting metabolites in original states contained in a urine sample as much as possible without modification thereof.

### DISCLOSURE

### Technical Problem

In order to extract metabolites in a urine sample in as large amounts as possible without modification thereof, the present inventors have established a urine metabolite extraction method using optimum extraction solvents without urease treatment and an analysis method of metabolites between different groups (e.g., sex, disease, etc.) based on the above metabolite extraction method, thereby completing the present invention.

Accordingly, it is an object of the present invention to provide a kit for discriminating sex (gender) by extracting metabolites from a urine sample.

Another object of the present invention is to provide a method for analyzing differences of metabolites between different groups in urine samples.

### Technical Solution

The present invention may provide a gender discrimination kit provided with a quantification device for one or more metabolites selected from the group consisting of succinate, fumarate, asparagine dihydrate, palmitic acid, β-alanine, L-cysteine, lactate, tyrosine, glycine and stearic acid.

Further, the present invention may provide,

a method for analyzing differences of metabolites between different groups in urine samples, including:

sampling a metabolite by extracting the metabolite with methanol or a solvent mixture of formic acid and methanol without urease treatment.

### Advantageous Effects

The present invention proposes an optimized extraction method of metabolites in a urine sample through non-urease treatment and comparison of extraction efficiency and extraction reproducibility between various extraction solvents in order to reproducibly extract sample as much of the metabolites in the urine as possible without change thereof. Further, a method for comparative analysis of metabolites between different groups based on the above extraction method is presented, thereby suggesting a method for detection of biomarkers such as gender, disease, etc.

The present invention is expected to be useful in various pathology and biomarker presentation studies through metabolite analysis of urine samples.

### DESCRIPTION OF DRAWINGS

FIG. 1 shows metabolite profiles (A: score plot, B: loading plot) between a stationary culture group (UI) at 37° C. for 1 hour with urease treatment using PLS-DA, another stationary culture group (WI) at 37° C. for 1 hour with non-urease treatment, and a non-stationary culture group (DE) with non-urease treatment.

FIG. 2 shows metabolite profiles (A: score plot, B: loading plot) between males (DE Male) and females (De-Female) in the non-stationary culture group (DE) with non-urease treatment using PLS-DA.

FIG. 3 illustrates comparison of amounts of 10 metabolites that distinguish males and females in a box plot.

FIG. 4 shows comparison box plots of metabolite extraction rates from urine on the basis of: pure methanol (MeOH); pure ethanol (EtOH); a mixture of acetonitrile:water (50 ACN; 1:1, v/v); and a mixture of water:2-propanol:methanol (WiPM; 2:2:5, v/v/v); and a mixture of formic acid:methanol (AM; 0.125:99.875, v/v).

FIG. 5 shows comparison box plots of variation coefficients (% CV) upon metabolite extraction from urine on the basis of: pure methanol (MeOH); pure ethanol (EtOH); a mixture of acetonitrile:water (50 ACN; 1:1, v/v); and a mixture of water:2-propanol:methanol (WiPM; 2:2:5, v/v/v); and a mixture of formic acid:methanol (AM; 0.125:99.875, v/v).

FIG. 6 shows comparison box plots (A) and photographs (B) of protein precipitations rates upon metabolite extraction from urine on the basis of: pure methanol (MeOH); pure ethanol (EtOH); a mixture of acetonitrile:water (50 ACN; 1:1, v/v); and a mixture of water:2-propanol:methanol (WiPM; 2:2:5, v/v/v); and a mixture of formic acid:methanol (AM; 0.125:99.875, v/v).

### BEST MODE

The present invention relates to a method for processing a urine sample for analysis of metabolites in urine.

According to an embodiment of the present invention, in order to reproducibly extract metabolites as much as possible in a urine sample without changes thereof, the metabolites may be directly extracted from the urine sample without urease treatment.

Further, according to another embodiment of the present invention, in order to propose a research method for distinguishing different groups based on metabolites of the urine sample and for finding biomarkers, different groups are compared and analyzed based on the metabolites extracted from the urine sample without urease treatment.

According to a further embodiment of the present invention, as large amounts as possible of the metabolites in urine may be reproducibly extracted, wherein pure methanol or a mixed solvent of formic acid and methanol may be used as an extraction solvent capable of extracting as large amounts of metabolites as possible in urine and properly precipitating proteins.

The present inventors have conducted extraction of metabolites using pure methanol or a mixed solvent of formic acid and methanol without urease treatment in order to find a biomarker that confirms discrimination between two biological sample groups in the urine sample, and comparative analysis of differences in metabolite profiles through GC/TOF/MS according to gender and pr-treatment methods of urine metabolites, followed by studies to discover desired biomarkers to distinguish gender using the above differences based on metabolites.

As a result, 107 and/or 113 metabolites including amines, amino acids, sugars and sugar alcohols, fatty acids, phosphoric acids, organic acids, and the like were identified.

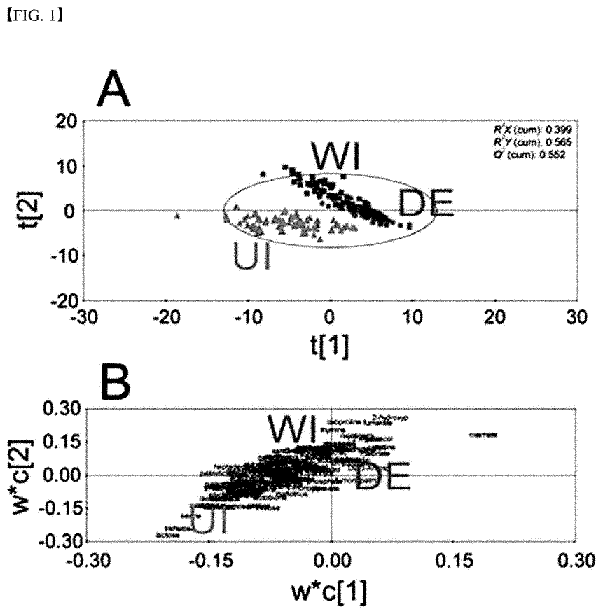

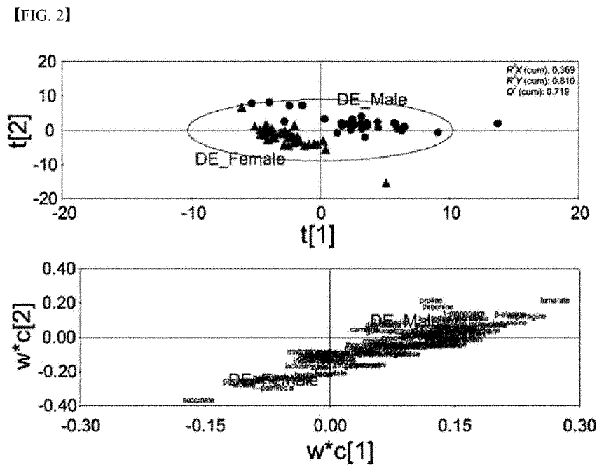

When comparing the biological samples from urine samples that were obtained different pre-treatment methods, a clear difference in metabolite profiles according to different pre-treatment methods by PLS-DA was confirmed (FIG. 1), and a difference in metabolite profiles in relation to gender was also clearly confirmed (FIG. 2).

Thereamong, in regard to gender discrimination models, top 10 metabolites were selected based on VIP value of PLS-DA model for each metabolite, which may be chosen as new biomarker candidates for gender discrimination (Table 4).

Therefore, the present invention may include a kit for gender identification which includes a quantification device for one or more metabolites selected from the group consisting of succinate, fumarate, asparagine dihydrate, palmitic acid, beta-alanine, L-cysteine, lactate, tyrosine, glycine and stearic acid.

Further, among metabolites in males, fumarate, asparagine dihydrate, β-alanine, L-cysteine and tyrosine tend to increase, while stearic acid, succinate, palmitic acid, lactic acid and glycine show a decreasing tendency.

Further, among the metabolites in females, succinate, palmitic acid, lactate, stearic acid and glycine tend to increase, while fumarate, asparagine dihydrate, β-alanine, L-cysteine and tyrosine show a decreasing tendency.

The increasing or decreasing tendency means an increase or decrease in concentrations of metabolites, and the term “increased metabolite concentration” means that the urine metabolite concentration of male to female or the urine metabolite concentration of female to male has increased significantly to be measurable. Likewise, in this specification, the term “decreased metabolite concentration” means that the urine metabolite concentration of female to male or the urine metabolite concentration of male to female has decreased significantly to be measurable.

The quantification device included in the kit of the present invention may be a chromatograph/mass spectrometer.

Chromatography used in the present invention may include, for example, gas chromatography, liquid-solid chromatography (LSC), paper chromatography (PC), thin-layer chromatography (TLC), gas-solid chromatography (GSC), liquid-liquid chromatography (LLC), foam chromatography (FC), emulsion chromatography (EC), gas-liquid chromatography (GLC), ion chromatography (IC), gel filtration chromatography (GFC), or gel permeation chromatography (GPC), but it is not limited thereto. In fact, all quantitative chromatography methods commonly used in the art may be used. Preferably, the chromatography used in the present invention is gas chromatography/time-of-flight mass spectrometry (GC/TOF MS).

With regard to the metabolite in the present invention, each component is separated by gas chromatography, and constitutional components thereof may be identified through structural information (elemental composition) as well as accurate molecular weight information using information obtained through TOF MS.

The present invention may also include a method for analysis of metabolite differentiation in urine to distinguish different groups.

According to one embodiment, the present invention may provide a method for analysis of metabolite differentiation in a urine sample to distinguish different groups (e.g., gender, disease, etc.).

Specifically, there is provided a method for analyzing differences of metabolites between different groups in urine samples, including sampling a metabolite by extracting the metabolite from a urine sample using pure methanol or a solvent mixture of formic acid and methanol without urease treatment.

The analysis method of metabolite differentiation may be a method of analyzing differentiation of metabolites in a urine sample between different groups, which includes a metabolite sampling step including: a quenching process; and a metabolite extraction process.

The metabolite sampling process may include extracting metabolites from the urine sample using pure methanol, pure ethanol, a mixture of acetonitrile:water; a mixture of water:2-propanol:methanol, or a mixture of formic acid:methanol without urease treatment. Specifically, the mixed solvent of formic acid:methanol is more preferably used. A mixing ratio of formic acid and methanol is more preferably a volume ratio of 0.05-0.5:99.5-99.95.

In this regard, the urine and extraction solvent are preferably treated in a volume ratio of 1:8 to 10 in order to reduce error in experiments.

The metabolites extracted in the metabolite sampling step may undergo the following analysis stages:

analyzing the extracted metabolites by means of a gas chromatograph/time-of-flight mass spectrometer (GC/TOF MS);

converting the GC/TOF MS analysis result into a numerical value capable of statistically processed; and

statistically verifying discrimination between different groups using the converted value.

Next, in order to compare a profiling difference in metabolites, partial least squares discriminant analysis (PLS-DA) was conducted to select metabolite biomarkers showing significant differences between different groups, so as to perform analysis and verification.

According to an embodiment, with regard to the analysis method of the present invention, the conversion of GC/TOF MS analysis results into statistically processable values may include dividing a total analysis time by unit time intervals, and determining the largest one of an area or height of chromatogram peaks displayed during the unit time as a representative value for the unit time.

The statistical verification of discrimination between two biological sample groups using the converted values may include analyzing and verifying metabolite biomarkers showing a significant difference between two biological sample groups through partial least squares discriminant analysis (PLS-DA).

The metabolite biomarkers according to an embodiment of the present invention may distinguish the gender of male and female.

The metabolite biomarkers may include succinate, fumarate, asparagine dihydrate, palmitic acid, β-alanine, L-cysteine, lactate, tyrosine, glycine and stearic acid.

A positive loading value of the partial least squares discriminant analysis (PLS-DA) indicates an increase in metabolite biomarkers, while a negative loading value indicates a decrease in metabolite biomarkers.

According to an embodiment of the present invention, biomarkers used herein for distinguishing gender may include one or more selected from the group consisting of succinate, fumarate, asparagine dihydrate, palmitic acid, β-alanine, L-cysteine, lactate, tyrosine, glycine and stearic acid.

Among the biomarkers, fumarate, asparagine dihydrate, β-alanine, L-cysteine and tyrosine tend to increase in males, while succinate, palmitic acid, lactate, stearic acid and glycine show a decreasing tendency in males.

On the other hand, among the biomarkers, succinate, palmitic acid, lactate, stearic acid and glycine tend to increase in females, while fumarate, asparagine dihydrate, β-alanine, L-cysteine and tyrosine show a decreasing tendency in females.

### DETAILED DESCRIPTION OF PREFERRED EMBODIMENTS OF INVENTION

Hereinafter, the present invention will be described in more detail through examples according to the present invention, but the scope of the present invention is not limited by the examples presented below.

### EXAMPLE

### Example 1: Metabolite Profiling of 68 Urine Samples Using PLS-DA

Urine samples obtained from 68 healthy adults (Table 1) were divided and treated as follows: a stationary culture group at 37° C. for 1 hour with urease treatment (UI); a stationary culture group at 37° C. for 1 hour without urease treatment (WI); and a non-stationary culture without urease treatment (DE), followed by extracting metabolites using pure methanol which has been widely used as an extraction solvent and then GC/TOF MS analysis.

107 metabolites were identified in the chemical classes of amines, amino acids, sugars and sugar alcohols, fatty acids and organic acids (Table 2).

In order to compare the profiling differences in metabolites, PLS-DA was conducted based on 106 metabolites except urea. With regard to the urease treatment and stationary culture group, the non-urease treatment and stationary culture treatment group, and the non-urease treatment and non-stationary group, respectively, different metabolite patterns were observed (FIG. 1, Tables 3 and 4). In other words, the metabolite profile of the urease treatment and stationary culture group was negative for most samples in terms of t[1] and t[2] values in a score plot. Likewise, the non-urease treatment and stationary culture group was positive for most samples in terms of t[1] and t[2] values in the score plot, while the non-urease treatment and non-stationary culture group had positive t[1] values and negative t[2] values for most samples. Briefly, it was confirmed that the metabolite profiles were completely distinguished according to the treatment methods (Table 3). Therefore, it could be demonstrated that the treatment methods, such as urease treatment or stationary culture, may modify or change other original metabolites in urine as well as urea.

Table 1 below shows urine sample information of 68 people.

Table 2 below shows 107 metabolites extracted from 68 urine samples using pure methanol.

Table 3 below shows t[1](PC1) and t[2](PC2) values represented as average and standard deviation (SD) in the metabolite profiles between: the stationary culture group at 37° C. for 1 hour with urease treatment using PLS-DA (UI); the stationary culture group at 37° C. for 1 hour with non-urease treatment (WI); and the non-urease-treatment and non-stationary culture group (DE).

Table 4 below shows loading values of the metabolites in the metabolite profiles between: the stationary culture group at 37° C. for 1 hour with urease treatment using PLS-DA (UI); the stationary culture group at 37° C. for 1 hour with non-urease treatment (WI); and the non-urease-treatment and non-stationary culture group (DE).

TABLE 1

| Adult male sample | Age | Adult female sample | Age |
|---|---|---|---|
| Male_l | M133 | Female_1 | F/32 |
| Male_2 | M/32 | Female_2 | F/36 |
| Male_3 | M/32 | Female_3 | F/37 |
| Male_4 | M/37 | Female_4 | F/34 |
| Male_5 | M/36 | Female_5 | F/37 |
| Male_6 | M/32 | Female_6 | F/39 |
| Male_7 | M/38 | Female_7 | F/39 |
| Male_8 | M/37 | Female_8 | F/38 |
| Male_9 | M/39 | Female_9 | F/34 |
| Male_10 | M/37 | Female_10 | F/37 |
| Male_11 | M/30 | Female_11 | F/36 |
| Male_12 | M/34 | Female_12 | F/38 |
| Male_13 | M/35 | Female_13 | F/39 |
| Male_14 | M/41 | Female_14 | F/36 |
| Male_15 | M/41 | Female_15 | F/45 |
| Male_16 | M/42 | Female_16 | F/44 |
| Male_17 | M/49 | Female_17 | F/47 |
| Male_18 | M/41 | Female_18 | F/48 |
| Male_19 | M/48 | Female_19 | F/43 |
| Male_20 | M/44 | Female_20 | F/42 |
| Male_21 | M/46 | Female_21 | F/40 |
| Male_22 | M/43 | Female_22 | F/46 |
| Male_23 | M/42 | Female_23 | F/42 |
| Male_24 | M/41 | Female_24 | F/41 |
| Male_25 | M/48 | Female_25 | F/43 |
| Male_26 | M/50 | Female_26 | F/53 |
| Male_27 | M/54 | Female_27 | F/50 |
| Male_28 | M/51 | Female_28 | F/51 |
| Male_29 | M/52 | Female_29 | F/50 |
| Male_30 | M/51 | Female_30 | F/51 |
| Male_31 | M/53 | Female_31 | F/51 |
| | | Female_32 | F/54 |
| | | Female_33 | F/53 |
| | | Female_34 | F/52 |
| | | Female_35 | F/52 |
| | | Female_36 | F/65 |
| | | Female_37 | F/63 |

TABLE 2
Identification of metaboiltes
Amines

2-hydroxypyridine
3-hydroxypyridine
5-deoxy-5-
methylthioadenosine
adenosne
benzamide
carnitine
glycocyamine
hypoxanthine
inosine
nicotinamide
O-phosphorylethanolamine
spermidine
thymine
tyrosine
uracil
urea
uric acid
uridine
xanthine

Ammo acids

alanine
asparagine dehydrated
glycine
histidine
isoleucine
L-allothreonine
L-cysteine
L-homoserine
lysine
methionine
methionine sulfoxide
N-methylalanine
ornithine
oxoproline
phenylalanine
proline
serine
threonine
tryptophan
valine
β-alanine

Fatty acids

1-monopalmitin
1-monostearin
arachidic acid
capric acid
heptadecanoic acid
lignoceric acid
myristic acid
palatinitol
palmitic acid
pelargonic acid
stearic acid

Organic acids

2-hydroxyvalerate
2-ketoadipate
3-hydroxypropionate
5-aminovalerate
adipate
aspartate
citramalate
citrate
DL-3-aminoisobutyrate
fumarate
galactonate
galacturonate
gluconic acid lactone
glycerate
glycolate
guaiacol
hexonate
indole-3-lactate
lactate
lactobionate
malate
malonate
oxalate
oxamate
pyrrole-2-carboxylate
pyruvate
succinate

Sugars and sugar alcohols

1,5-anhydroglucitol
3,6-anhydro-D-
galactose
arabitol
dihydoxyacetone
fructose
glycerol
galactinol
galactose
glucose
glycerol-1-phosphate
lactose
lyxose
maltotriose
mannitol
mannose
melibiose
myo-inositol
ribose
sucrose
tagatose
threitol
threose
trehalose
xylose

Miscellaneous

1,2,4-benzenetriol
caffeic acid
phosphate
taurine
xanthurenic acid

TABLE 3

| Class | t[1]_average | t[2]_average | t[1]_stdev | t[2]_stdev |
|---|---|---|---|---|
| DE | 4.368 | −0.401 | 2,117 | 1.687 |
| WI | 0.837 | 3231 | 3.376 | 3.703 |
| UI | −5.257 | −2.776 | 4.334 | 1.468 |

Table 3 shows that types and amounts of the metabolites may vary depending upon treatment. It could be assumed that the metabolites may be extracted from the DE group without any pre-treatment, thereby maintaining the original types and amounts of metabolites in urine. The urease treatment and stationary culture group at 37° C. for 1 hour (UI) and the non-urease treatment and stationary culture group at 37° C. for 1 hour (WI) had changed t[1] values or t[2] values in most samples, thereby demonstrating variation in types and amounts of the metabolites (FIG. 1, Table 3). Changes in the type and amount of metabolites through such treatment were found result in changes of the type or amount of biomarker substances for diagnosis of diseases, reduce the ability to discover biomarkers, and as a result false biomarkers may be selected.

Therefore, since the urease treatment changes the metabolite profile (Table 3), a biomarker discovering ability is lower than that of the non-urease treatment group DE having intrinsic metabolite profile.

TABLE 4

| Metabolite | Loading 1 | Loading 2 |
|---|---|---|
| 1,2,4-benzenetriol | 0.015 | 0.174 |
| 1,5-anhydroglucitol | −0.043 | 0.020 |
| 1-monopalmitin | 0.001 | −0.013 |
| 1-monostearin | −0.058 | −0.047 |
| 2-hydroxypyridine | 0.048 | 0.269 |
| 2-hydroxyvalerate | −0.111 | −0.025 |
| 2-ketoadipate | 0.001 | 0.078 |
| 3,6-anhydro-D-galactose | −0.102 | 0.052 |
| 3-hydroxypropionate | −0.134 | −0.067 |
| 3-hydroxypyridine | −0.058 | 0.120 |
| 5-aminovalerate | −0.055 | 0.037 |
| 5′-deoxy-5′-methylthioadenosine | −0.130 | −0.067 |
| Adenosine | −0.128 | −0.004 |
| Adipate | −0.035 | 0.095 |
| Alanine | −0.085 | 0.067 |
| arabitol | −0.019 | 0.122 |
| arachidic acid | −0.080 | 0.072 |
| asparagine dehydrate | −0.076 | −0.010 |
| aspartate | −0.076 | 0.029 |
| benzamide | −0.031 | 0.134 |
| O-alanine | −0.043 | 0.035 |
| caffeic acid | −0.023 | 0.112 |
| capric acid | −0.160 | −0.135 |
| carnitine | 0.041 | 0.135 |
| citramalate | −0.098 | 0.035 |
| citrate | 0.009 | 0.080 |
| dihydroxyacetone | −0.002 | 0.064 |
| DL-3-aminoisobutyrate | 0.018 | 0.041 |
| fructose | −0.052 | 0.059 |
| fumarate | 0.037 | 0.246 |
| galactinol | −0.069 | −0.007 |
| galactonate | −0.158 | −0.032 |
| galactose | −0.093 | 0.090 |
| galacturonate | −0.159 | −0.044 |
| gluconic acid lactone | −0.084 | 0.025 |
| glucose | −0.111 | 0.057 |
| glycerate | −0.075 | −0.032 |
| glycerol | −0.153 | −0.080 |
| glycerol-1-phosphate | −0.043 | 0.129 |
| glycine | 0.048 | 0.136 |
| glycocyamine | −0.090 | 0.070 |
| glycolate | −0.130 | −0.046 |
| guaiacol | 0.038 | 0.175 |
| heptadecanoic acid | −0.147 | 0.047 |
| hexonate | −0.152 | −0.096 |
| histidine | −0.104 | −0.057 |
| hypoxanthine | −0.045 | 0.135 |
| indole-3-lactate | −0.075 | 0.026 |
| inosine | −0.080 | −0.001 |
| isoleucine | −0.165 | −0.095 |
| lactate | −0.076 | 0.010 |
| lactobionate | −0.099 | −0.090 |
| lactose | −0.218 | −0.261 |
| L-allothreonine | −0.009 | 0.147 |
| L-cysteine | −0.101 | 0.009 |
| L-homoserine | −0.143 | −0.131 |
| lignoceric acid | −0.092 | −0.041 |
| lysine | −0.066 | 0.014 |
| lyxose | −0.052 | 0.063 |
| malate | −0.060 | 0.076 |
| malonate | 0.032 | 0.108 |
| maltotriose | −0.071 | −0.076 |
| mannitol | −0.026 | 0.036 |
| mannose | −0.047 | 0.134 |
| melibiose | −0.063 | −0.008 |
| methionine | −0.131 | −0.065 |
| methionine sulfoxide | −0.132 | 0.026 |
| myo-inositol | −0.049 | 0.048 |
| myristic acid | −0.112 | 0.034 |
| nicotinamide | 0.008 | 0.211 |
| N-methylalanine | −0.137 | −0.078 |
| O-phosphorylethanolamine | −0.129 | −0.048 |
| ornithine | −0.083 | 0.015 |
| oxalate | −0.177 | −0.128 |
| oxamate | 0.166 | 0.193 |
| oxoproline | −0.008 | 0.252 |
| palatinitol | −0.128 | −0.080 |
| palmitic acid | −0.164 | 0.019 |
| pelargonic acid | −0.127 | −0.018 |
| phenylalanine | −0.137 | −0.030 |
| phosphate | −0.033 | −0.019 |
| proline | −0.105 | −0.128 |
| pyrrole-2-carboxylate | −0.074 | −0.017 |
| pyruvate | −0.027 | −0.051 |
| ribose | −0.131 | 0.015 |
| serine | −0.187 | −0.175 |
| spermidine | 0.006 | 0.126 |
| stearic acid | −0.013 | 0.152 |
| succinate | −0.042 | 0.103 |
| sucrose | −0.166 | −0.127 |
| tagatose | −0.052 | 0.045 |
| taurine | −0.087 | −0.019 |
| threitol | −0.010 | 0.079 |
| threonine | 0.006 | 0.126 |
| threose | −0.096 | −0.138 |
| thymine | −0.016 | 0.214 |
| trehalose | −0.207 | −0.232 |
| tryptophan | −0.099 | 0.019 |
| tyrosine | −0.086 | 0.046 |
| uracil | −0.068 | 0.070 |
| uric acid | −0.037 | 0.077 |
| uridine | −0.096 | 0.036 |
| valine | −0.150 | −0.051 |
| xanthine | −0.076 | 0.118 |
| xanthurenic acid | −0.084 | 0.093 |
| xylose | −0.125 | −0.033 |

### Example 2: Selection of Major Metabolites in 68 Urine Samples

Using the PES-DA analysis from Example 1, the top 10 major metabolites contributing greatly to classification of 68 urine samples into three (3) groups, that is: a stationary culture group at 37° C. for 1 hour with urease treatment using PLS-DA (UI); a stationary culture group at 37° C. for 1 hour with non-urease treatment (WI); and a non-urease-treatment and non-stationary culture group (DE), were selected with reference to VIP (variable importance in projection) score values (Table 5).

Table 5 below shows VIP score values of the 10 major metabolites that have high differences in metabolite profiles between: the stationary culture group at 37° C. for 1 hour with urease treatment using PLS-DA (UI); the stationary culture group at 37° C. for 1 hour with non-urease treatment (WI); and the non-urease-treatment and non-stationary culture group (DE).

TABLE 5

| Metabolites | VIP value |
|---|---|
| Succinate | 2.650 |
| palmitic acid | 2.468 |
| 1-monostearin | 2.093 |
| 1-monopalmitin | 1.873 |
| Benzamide | 1.786 |
| heptadecanoic acid | 1.724 |
| Malate | 1.696 |
| O-alanine | 1.632 |
| Histidine | 1.573 |
| gluconic acid lactone | 1.567 |

### Example 3: Metabolite Profiling to Distinguish Male and Female of 68 Urine Samples Using PLS-DA

Among the urine samples obtained from 68 healthy adults (Table 1), 31 male urine samples and 37 female urine samples were extracted without urease treatment and metabolites were extracted using pure methanol which has been previously used, as an extraction solvent, followed by analysis through GC/TOF MS. Thereafter, a PLS-DA model was prepared using 106 metabolites excluding urea, so as to distinguish the gender (FIG. 2, Tables 6 and 7).

As shown in FIG. 2, metabolites in urine of males and females have different patterns, and statistically significant differences were shown based on the PLS-DA model. That is, the metabolite profile for male classification was positive in the score plot for most samples in terms of t[1] and t[2] values, and the metabolite profile for female classification was negative in the score plot for most samples in terms of [t]1 and t[2] values, thereby demonstrating that the metabolite profiles in relation to the gender were completely distinguished (Table 7). In order to select the major metabolites showing a difference in metabolite profiles, metabolites having the same trend in both loading 1 and loading 2 in Table 8 were selected.

Table 6 below shows the average and standard deviation of the t[1] and t[2] values of each sample in the metabolite profile that shows a difference in metabolite profiling to distinguish males and females from 68 urine samples using PLS-DA.

Table 7 below shows the loading values of each metabolite in the metabolite profile that shows a difference in metabolite profiling to distinguish males and females from 68 urine samples using PLS-DA.

TABLE 6

| Class | t[1]_average | t[2]_average | t[1]_stdev | t[2]_stdev |
|---|---|---|---|---|
| Male | −3.0..54 | −2.210 | 3.821 | 2485 |
| Female | 2.558 | 1.852 | 1.981 | 1231 |

TABLE 7

| Metabolite | Loading 1 | Loading 2 |
|---|---|---|
| 1,2,4-benzenetriol | 0.070 | −0.043 |
| 1,5-anhydroglucitol | −0.043 | −0.120 |
| 1-monopalmitin | 0.132 | 0.155 |
| 1-monostearin | 0.140 | 0.161 |
| 2-hydroxypyridine | 0.082 | −0.028 |
| 2-hydroxyvalerate | 0.047 | −0.038 |
| 2-ketoadipate | 0.047 | 0.105 |
| 3,6-anhydro-D-galactose | 0.110 | −0.011 |
| 3-hydroxypropionate | −0.068 | −0.229 |
| 3-hydroxypyridine | 0.103 | 0.012 |
| 5-aminovalerate | 0.055 | −0.034 |
| 5′-deoxy-5′-methylthioadenosine | −0.015 | −0.123 |
| adenosine | 0.111 | 0.004 |
| Adipate | 0.009 | −0.074 |
| Alanine | 0.119 | 0.031 |
| arabitol | 0.122 | 0.071 |
| arachidic acid | 0.046 | −0.030 |
| asparagine dehydrate | 0.210 | 0.139 |
| aspartate | −0.022 | −0.195 |
| benzamide | 0.023 | −0.075 |
| O-alanine | 0.195 | 0.149 |
| caffeic acid | −0.015 | −0.073 |
| capric acid | 0.116 | 0.058 |
| camitine | 0.021 | 0.060 |
| citramalate | 0.091 | −0.019 |
| Citrate | −0.111 | −0.236 |
| dihydroxyacetone | 0.039 | 0.089 |
| DL-3-aminoisobutyrate | 0.079 | 0.019 |
| fructose | 0.006 | −0.151 |
| fumarate | 0.250 | 0.231 |
| galactinol | −0.040 | −0.102 |
| galactonate | 0.015 | −0.149 |
| galactose | 0.110 | −0.030 |
| galacturonate | 0.094 | −0.019 |
| gluconic acid lactone | 0.064 | −0.038 |
| Glucose | 0.073 | −0.083 |
| glycerate | −0.022 | −0.096 |
| glycerol | −0.056 | −0.228 |
| glycerol-1-phosphate | 0.101 | 0.044 |
| Glycine | −0.132 | −0.242 |
| glycocyamine | 0.019 | −0.146 |
| glycolate | 0.074 | −0.043 |
| guaiacol | 0.040 | 0.053 |
| heptadecanoic acid | −0.045 | −0.203 |
| hexonate | 0.060 | 0.010 |
| histidine | 0.138 | 0.059 |
| hypoxanthine | 0.134 | 0.041 |
| indole-3-lactate | −0.047 | −0.124 |
| Inosine | −0.020 | −0.095 |
| isoleucine | 0.164 | 0.096 |
| Lactate | −0.119 | −0.262 |
| lactobionate | −0.070 | −0.219 |
| Lactose | −0.057 | −0.153 |
| L-allothreonine | 0.104 | 0.128 |
| L-cysteine | 0.192 | 0.102 |
| L-homoserine | 0.044 | −0.046 |
| lignoceric acid | 0.100 | 0.081 |
| Lysine | 0.098 | 0.031 |
| Lyxose | 0.082 | 0.034 |
| Malate | −0.093 | −0.227 |
| malonate | 0.125 | 0.088 |
| maltotriose | −0.054 | −0.071 |
| mannitol | 0.126 | 0.112 |
| Mannose | 0.048 | −0.088 |
| melibiose | −0.041 | −0.092 |
| methionine | 0.157 | 0.057 |
| methionine sulfoxide | 0.117 | −0.028 |
| myo-inositol | 0.048 | −0.023 |
| myristic acid | −0.034 | −0.152 |
| nicotinamide | 0.131 | 0.012 |
| N-methylalanine | 0.128 | 0.068 |
| O-phosphorylethanolamine | 0.096 | 0.049 |
| ornithine | −0.009 | −0.113 |
| Oxalate | 0.036 | −0.008 |
| Oxamate | 0.066 | 0.051 |
| oxoproline | 0.114 | −0.034 |
| palatinitol | 0.015 | −0.052 |
| palmitic acid | −0.086 | −0.281 |
| pelargonic acid | −0.096 | −0.220 |
| phenylalanine | 0.136 | 0.001 |
| phosphate | −0.026 | −0.094 |
| Proline | 0.105 | 0.232 |
| pyrrole-2-carboxylate | 0.049 | −0.026 |
| pyruvate | 0.023 | −0.055 |
| Ribose | 0.064 | −0.071 |
| Serine | 0.107 | 0.029 |
| spermidine | 0.088 | 0.027 |
| stearic acid | −0.107 | −0.242 |
| succinate | −0.180 | −0.353 |
| Sucrose | −0.034 | −0.069 |
| tagatose | 0.023 | −0.079 |
| Taurine | 0.051 | −0.025 |
| threitol | 0.068 | −0.083 |
| threonine | 0.107 | 0.193 |
| Threose | 0.016 | −0.031 |
| Thymine | 0.153 | 0.075 |
| trehalose | −0.018 | −0.078 |
| tryptophan | 0.154 | 0.049 |
| tyrosine | 0.178 | 0.088 |
| Uracil | −0.007 | −0.126 |
| uric acid | 0.146 | 0.131 |
| Uridine | 0.135 | −0.002 |
| Valine | 0.115 | 0.012 |
| xanthine | 0.017 | −0.088 |
| xanthurenic acid | 0.143 | 0.034 |
| Xylose | −0.026 | −0.162 |

### Example 4: Selection of Major Metabolites Showing Differences in Metabolite Profiling that Distinguishes Males and Females from 68 Urine Samples Using PLS-DA

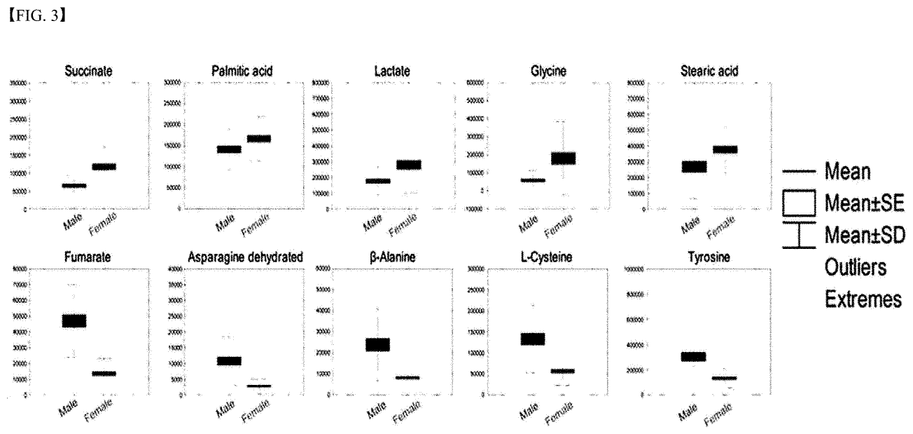

Using the PLS-DA analysis from Example 3, it was confirmed that each gender group was separated, and the top to major metabolites showing high VIP values, which are a degree of contribution to the separation of gender in the model, were selected. (Table 8). Further, the amounts of 10 major metabolites were indicated in a box plot to compare the same with the amounts of metabolites according to gender (FIG. 3).

Next, Table 8 below shows VIP (variable importance in projection) score values of the 10 major metabolites having have significant differences in metabolite profiles that show a difference in metabolite profiling to distinguish males and females from 68 urine samples using PLS-DA.

TABLE 8

| Metabolite | VIP score |
|---|---|
| succinate | 2.045 |
| fumarate | 2.003 |
| asparagine dehydrate | 1.666 |
| palmitic acid | 1.595 |
| O-alanine | 1.541 |
| L-cysteine | 1.540 |
| lactate | 1.494 |
| tyrosine | 1.432 |
| glycine | 1.420 |
| stearic acid | 1.373 |

### Example 5: Selection of the Optimal Extraction Solvent for Analysis of Metabolites in Urine Samples

In order to obtain metabolite samples from urine samples, 68 urine samples were combined in equal proportions to form a urine mixture, and then, 100 μl of the urine mixture was directly treated with 900 μl of extraction solvent, that is: pure methanol (MeOH); pure ethanol (EtOH); a mixture of acetonitrile:water (50 ACN; 1:1, v/v); a mixture of water:2-propanol/methanol (WiPM; 2:2:5, v/v/v); and a mixture of formic acid:methanol (AM; 0.125:99.875, v/v), respectively, without urease treatment, so as to extract metabolites, followed by GC/TOF-MS analysis to compare and analyze extraction efficiencies thereof.

In the urine mixture, 113 metabolites including amines, amino acids, sugars and sugar alcohols, fatty acids, and organic acids were identified (Table 9).

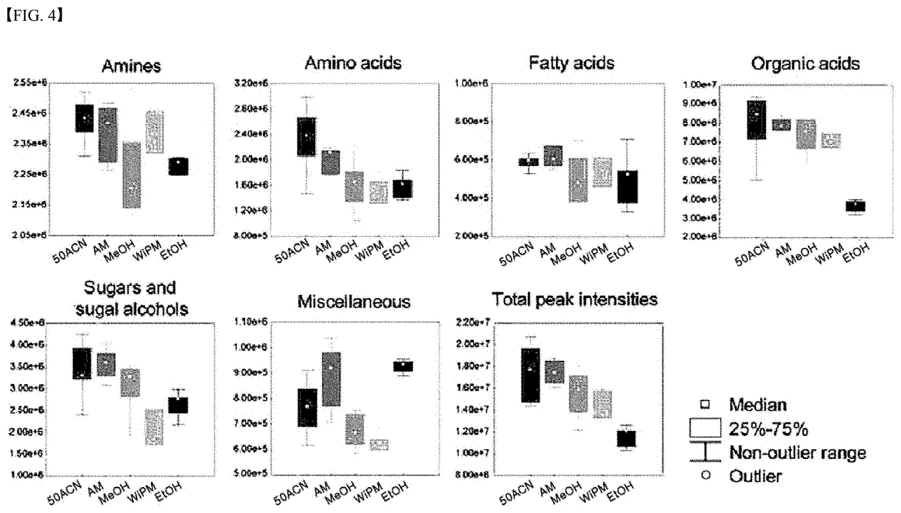

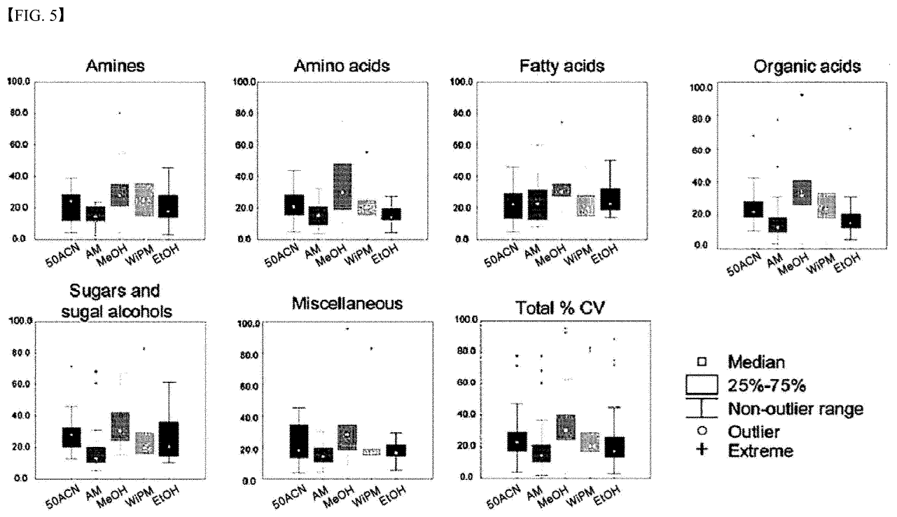

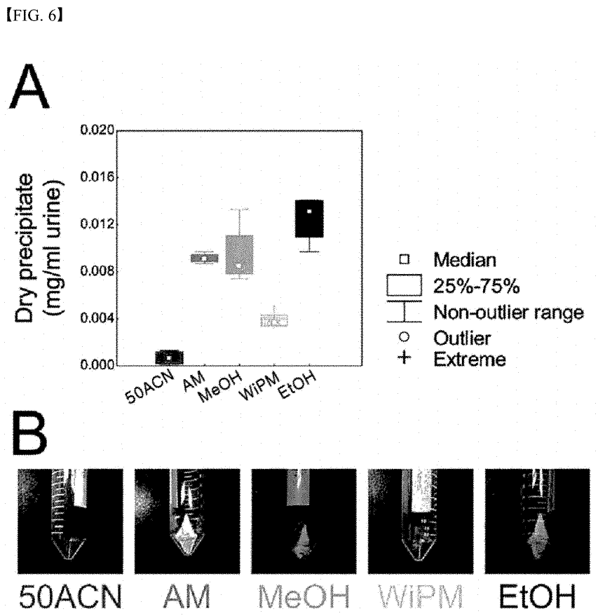

As shown in FIGS. 4 and 5, it was confirmed that the extraction rate and extraction reproducibility were different depending on the extraction solvent. It could be seen that the peak intensity analyzed qualitatively and relatively quantitatively was the highest in AM, thereby demonstrating the highest extraction rate of comprehensive metabolites in AM (FIG. 4). Further, with regard to reproducibility according to the extraction solvent, it was found that the % CV value recorded the lowest value in both AM, thereby demonstrating the highest reproducibility (FIG. 5). Further, the protein sedimentation rate recorded the second highest value in AM, thereby demonstrating appropriate protein sedimentation ability of AM (FIG. 6). According to the above results. AM was selected as the optimal solvent based on the extraction rate, reproducibility and protein precipitation rate when metabolites are extracted for metabolite analysis in urine.

Table 9 below shows 113 metabolites extracted from a human urine mixture sample using: pure methanol; pure ethanol; a mixture of acetonitrile:water; a mixture of water:2-propanol:methanol; and a mixture of formic acid:methanol, respectively.

TABLE 9
Identification of metaboiltes
Amines

2-hydroxypyridine
3-hydroxypyridine
5-deoxy-5-
methylthioadenosine
adenosne
benzamide
carnitine
glycocyamine
hypoxanthine
inosine
nicotinamide
O-phosphorylethanolamine
spermidine
thymine
tyrosine
uracil
urea
uric acid
uridine
xanthine

Ammo acids

alanine
asparagine dehydrated
glycine
histidine
isoleucine
L-allothreonine
L-cysteine
L-homoserine
lysine
methionine
methionine sulfoxide
N-methylalanine
ornithine
oxoproline
phenylalanine
proline
serine
threonine
tryptophan
valine
β-alanine

Fatty acids

1-monopalmitin
1-monostearin
arachidic acid
capric acid
heptadecanoic acid
lignoceric acid
myristic acid
palatinitol
palmitic acid
pelargonic acid
stearic acid

Organic acids

2-hydroxyvalerate
2-ketoadipate
3-hydroxypropionate
5-aminovalerate
adipate
aspartate
citramalate
citrate
DL-3-aminoisobutyrate
fumarate
galactonate
galacturonate
gluconic acid lactone
glycerate
glycolate
guaiacol
hexonate
indole-3-lactate
lactate
lactobionate
malate
malonate
oxalate
oxamate
pyrrole-2-carboxylate
pyruvate
succinate

Sugars and sugar alcohols

1,5-anhydroglucitol
3,6-anhydro-D-
galactose
arabitol
dihydoxyacetone
fructose
glycerol
galactinol
galactose
glucose
glycerol-1-phosphate
lactose
lyxose
maltotriose
mannitol
mannose
melibiose
myo-inositol
ribose
sucrose
tagatose
threitol
threose
trehalose
xylose

Miscellaneous

1,2,4-benzenetriol
caffeic acid
phosphate
taurine
xanthurenic acid

1. A gender discrimination kit, comprising a quantitative device for one or more urine metabolites selected from the group consisting of succinate, fumarate, asparagines dihydrate, palmitic acid, β-alanine, L-cysteine, lactate, tyrosine, glycine and stearic acid.

2. The kit according to claim 1, wherein the quantitative device is a gas chromatography/time-of-flight mass spectrometry (GC/TOF MS) analyzer.

3. The kit according to claim 1, wherein, in the case of males, fumarate, asparagines dihydrate, β-alanine, L-cysteine and tyrosine among the above metabolites tend to increase while succinate, palmitic acid, lactate, stearic acid and glycine have a decreasing tendency.

4. The kit according to claim 1, wherein, in the case of females, succinate, palmitic acid, lactate, stearic acid and glycine among the above metabolites tend to increase while fumarate, asparagines dihydrate, β-alanine, L-cysteine and tyrosine have a decreasing tendency.

5. A method for analysis of metabolite differentiation between different groups in urine samples, comprising:
a metabolite sampling step that extracts metabolites from urine using pure methanol or a mixed solvent of formic acid and methanol without urease treatment of the urine.

6. The method according to claim 5, further comprising:
analyzing the extracted metabolites by means of the GC/TOF MS analyzer;
converting the GC/TOF MS analysis result into a numerical value capable of statistically processed; and
statistically verifying discrimination between different groups using the converted value.

7. The method according to claim 5, wherein the converting step of the GC/TOF MS analysis result into a numerical value capable of statistically processing includes dividing a total analysis time by unit time intervals, and determining the largest one of an area or height of chromatogram peaks displayed during the unit time as a representative value for the unit time.

8. The method according to claim 5, wherein the statistical verifying step of discrimination between two biological sample groups using the converted values includes conducting partial least squares discriminant analysis (PLS-DA) so as to analyze and verify metabolite biomarkers that show a significant difference between these two biological sample groups.

9. The method according to claim 8, wherein a positive loading value of the partial least squares discriminant analysis (PLS-DA) indicates an increasing tendency of metabolite biomarkers, while a negative loading value indicates a decreasing tendency of metabolite biomarkers.

10. The method according to claim 8, wherein the metabolite biomarkers consist of succinate, fumarate, asparagines dihydrate, palmitic acid, β-alanine, L-cysteine, lactate, tyrosine, glycine and stearic acid.

11. The method according to claim 8, wherein the metabolite biomarkers discriminate gender.
